(19) United States
(12) Patent Application Publication
(10) Pub. No.: US 2025/0360333 A1
(43) Pub. Date: Nov. 27, 2025

(54) METHOD, DEVICE AND RECORDING MEDIUM FOR PROVIDING THERAPY THROUGH BRAIN WAVE DATA ANALYSIS

(71) Applicant: iMediSync, Inc., Seoul (KR)

(72) Inventors: Seungwan KANG, Seoul (KR); Dae Keun KIM, Seoul (KR)

(73) Assignee: iMediSync, Inc., Seoul (KR)

(21) Appl. No.: 19/289,719

(22) Filed: Aug. 4, 2025

Related U.S. Application Data
(63) Continuation of application No. PCT/KR2024/001990, filed on Feb. 13, 2024.

(30) Foreign Application Priority Data
Feb. 14, 2023 (KR) 10-2023-0019239
Jun. 1, 2023 (KR) 10-2023-0070634

(51) Int. Cl. A61N 5/06

(52) U.S. Cl. CPC A61N 5/0622; A61N 2005/0626; A61N 2005/0659; A61N 2005/0663

(57) ABSTRACT

A method, device and recording medium for providing therapy through brain wave data analysis are provided. The method by which a computing device provides therapy through brain wave data analysis, according to various embodiments of the present invention, comprises the steps of: acquiring brain wave data of a user; determining a therapy protocol on the basis of the acquired brain wave data; and using the determined therapy protocol to provide therapy to the user.

### CROSS-REFERENCE TO RELATED PATENT APPLICATION

This application is a Continuation of International Application No. PCT/KR2024/001990, filed on Feb. 13, 2024, which claims the benefit of Korean Patent Application Nos. 10-2023-0019239, filed on Feb. 14, 2023, and 10-2023-0070634, filed on Jun. 1, 2023, in the Korean Intellectual Property Office, the disclosure of which is incorporated herein in its entirety by reference.

### TECHNICAL FIELD

The present invention relates to a method, a device, and a recording medium for providing therapy through brain wave analysis, and more particularly, to a method, a device, and a recording medium for providing therapy through brain wave analysis that analyze a user's brain wave data to provide optimized therapy.

### BACKGROUND ART

Although pharmacologic treatments are provided for several brain disorders, there are many opinions that pharmacologic treatment alone is insufficient. In addition to pharmacologic treatment, several neurostimulation methods are under research.

Traditional neurostimulation methods include transcranial magnetic stimulation (tMS), transcranial alternating current stimulation (tACS), transcranial direct current stimulation (tDCS), transcranial random noise stimulation (tRNS), photobiomodulation (PBM), and the like.

Here, PBM methods are based on the principle of using light mainly with infrared wavelengths to cause photochemical changes inside the mitochondrial cell structure.

More specifically, the biochemical mechanism of an interaction of a PBM method may have direct effects and indirect effects. The direct effects include an increase in ion channel activity, such as Na+/K+ adenosine triphosphatase (ATPase), and the indirect effects include regulation of important second messengers such as calcium, cyclic adenosine monophosphate (cAMP), and reactive oxygen species. Both effects result in various biological cascades. These biological cascades lead to not only effects such as homeostasis maintenance and protection and activation of antioxidant and proliferative genetic factors but also hierarchical responses such as cerebral blood flow with less neurocognitive impairment.

However, there has been no technology for proposing such adjustment methods appropriate for a user's condition.

### DISCLOSURE

### Technical Problem

An object to be solved by the present invention is to provide a method, a device, and a recording medium for providing therapy through brain wave analysis that analyze a user's brain wave data to determine a therapy protocol and provide therapy to the user on the basis of the therapy protocol such that optimal therapy, that is, therapy for deriving appropriate brain waves for the user, may be provided in consideration of the user's current condition or brain disease.

Objects to be solved by the present invention are not limited to that described above, and other objects that have not been described will be clearly understood by those skilled in the art from the following description.

### Technical Solution

To achieve the above-described purpose, a method of providing therapy through brain wave data analysis performed by a computing device according to an embodiment of the present invention includes acquiring a user's brain wave data, determining a therapy protocol on the basis of the acquired brain wave data, and providing therapy to the user using the determined therapy protocol.

According to various embodiments, the determining of the therapy protocol may include analyzing the acquired brain wave data to determine necessity of providing therapy to the user and determining a therapy protocol for the user when it is determined that it is necessary to provide therapy to the user. The determined therapy protocol may include at least one of sessions, a frequency, a treatment area, a duration, and a light wavelength of the therapy to be provided to the user.

According to various embodiments, the determining of the therapy protocol may include specifying an abnormal region on the basis of the acquired brain wave data and determining a frequency and a duration of the therapy for leading a brain wave corresponding to the specified abnormal region to a preset reference value as a therapy protocol for the user.

According to various embodiments, the specifying of the abnormal region may include setting a target and comparing position-specific brain wave values included in the acquired brain wave data with position-specific brain wave reference values in accordance with the set target to specify the abnormal region.

According to various embodiments, the determining of the therapy protocol may include determining a treatment area of the therapy as the therapy protocol for the user by analyzing the acquired brain wave data.

According to various embodiments, the determining of the treatment area may include, when the user is diagnosed with a specific brain disease by analyzing the acquired brain wave data, determining a region in which the specific brain disease is diagnosed as a first treatment area and determining a region related to treatment of the diagnosed specific brain disease as a second treatment area.

According to various embodiments, the determining of the therapy protocol may include, when an abnormal region of the user is specified by analyzing the acquired brain wave data, determining the therapy protocol such that the therapy may be provided to the specified abnormal region alone.

According to various embodiments, the determining of the therapy protocol may include, when two or more abnormal regions of the user are specified by analyzing the acquired brain wave data, separately determining therapy protocols corresponding to each of the two or more specified abnormal regions.

According to various embodiments, the determining of the therapy protocol may include specifying an abnormal region of the user by analyzing the acquired brain wave data, calculating an indicator corresponding to a degree of abnormality of the specified abnormal region on the basis of a brain wave corresponding to the specified abnormal region, and determining a frequency of the therapy as the therapy protocol for the user.

According to various embodiments, the acquiring of the brain wave data may include acquiring the user's brain wave data measured through a plurality of channels included in a brain wave measurement device in accordance with a preset measurement protocol. Distances between the plurality of channels may be adjusted in accordance with the user's head size.

To achieve the above-described purpose, a computing device for performing a method of providing therapy through brain wave data analysis according to another embodiment of the present invention includes a processor, a network interface, a memory, and a computer program that is loaded into the memory and executed by the processor. The computer program may include an instruction to acquire a user's brain wave data, an instruction to determine a therapy protocol on the basis of the acquired brain wave data, and an instruction to provide therapy to the user using the determined therapy protocol.

To achieve the above-described purpose, a computer program according to still another embodiment of the present invention is stored in a recording medium which is readable by a computing device, to perform a method of providing therapy through brain wave data analysis in combination with a computing device, the method including acquiring a user's brain wave data, determining a therapy protocol on the basis of the acquired brain wave data, and providing therapy to the user using the determined therapy protocol.

Other details of the present invention are included in the detailed description and drawings.

### Advantageous Effects

According to various embodiments of the present invention, a user's brain wave data is analyzed to determine a therapy protocol, and therapy is provided to the user on the basis of the therapy protocol such that optimal therapy, that is, therapy for deriving appropriate brain waves for the user, can be provided in consideration of the user's current condition or brain disease.

Effects of the present invention are not limited to that described above, and other effects that have not been described will be clearly understood by those skilled in the art from the following description.

### DESCRIPTION OF DRAWINGS

FIG. 1 is a diagram showing a system for providing therapy through brain wave data analysis according to an embodiment of the present invention.

FIG. 2 is a hardware configuration diagram of a computing device that performs a method of providing therapy through brain wave data analysis according to another embodiment of the present invention.

FIG. 3 is a flowchart of a method of providing therapy through brain wave data analysis according to still another embodiment of the present invention.

FIGS. 4 and 5 are instrumental configuration views of a therapy provision device for providing therapy according to various embodiments of the present invention.

FIG. 6 is a view of a brain wave measurement and therapy provision module included in a therapy provision device according to various embodiments of the present invention.

FIGS. 7 and 8 are sets of brain wave views for comparing brain waves before and after therapy is provided using a method of providing therapy through brain wave data analysis according to various embodiments of the present invention.

FIG. 9 is a diagram illustrating an execution screen of an application provided by a computing device according to various embodiments of the present invention.

### MODES OF THE INVENTION

The advantages and features of the present invention and a method of achieving them will become apparent from embodiments which will be described in detail below with reference to the accompanying drawings. However, the present invention is not limited to the embodiments disclosed below and may be implemented in various different forms. The embodiments are only provided to make the disclosure of the present invention complete and fully convey the scope of the present invention to those skilled in the technical field to which the present invention pertains. The present invention is only defined by the scope of the claims.

Terminology used herein is only for the purpose of describing embodiments and is not intended to limit the present invention. In this specification, singular forms include plural forms as well unless the context particularly indicates otherwise. The terms “comprises” and/or “comprising” used in this specification do not preclude the presence or addition of one or more components other than stated components. Throughout the specification, like reference numerals refer to like components, and the term “and/or” includes any of stated components or a combination of one or more stated items. Although the terms “first,” “second,” and the like may be used to describe various components, these components are not limited by the terms. The terms are used for the sole purpose of distinguishing one component from others. Therefore, a first component mentioned below may be a second component within the technical scope of the present invention.

Unless defined otherwise, all terms used herein (including technical or scientific terms) have the same meanings as those generally understood by those skilled in the technical field to which the present invention pertains. In addition, terms defined in commonly used dictionaries are not interpreted ideally or excessively unless particularly defined herein.

As used herein, the term “unit” or “module” refers to a software component or a hardware component such as a field programmable gate array (FPGA) or an application-specific integrated circuit (ASIC), and a “unit” or “module” performs certain roles. However, a “unit” or “module” is not limited to software or hardware. A “unit” or “module” may be configured to reside in an addressable storage medium or run on one or more processors. Accordingly, as an example, a “unit” or “module” includes components, such as software components, object-oriented software components, class components, and task components, processes, functions, attributes, procedures, subroutines, segments of program code, drivers, firmware, microcode, circuitry, data, databases, data structures, tables, arrays, and variables. Components and functions provided in “units” or “modules” may be combined into a smaller number of components and “units” or “modules” or subdivided into additional components and “units” or “modules.”

Spatially relative terms “below,” “beneath,” “lower,” “above,” “upper,” and the like may be used to easily describe the relationship between a certain component and other components shown in the drawings. Spatially relative terms should be understood as terms that include different directions of components during use or operation in addition to directions shown in the drawings. For example, when a component shown in the drawings is turned over, the component described as “below” or “beneath” another component may be placed “above” the other component. Accordingly, the exemplary term “below” may include both directions, below and above. Components may also be oriented in other directions, and spatially relative terms may be interpreted according to orientation.

In this specification, a computer is any type of hardware device including at least one processor and may be understood as collectively including software configurations operating in a corresponding hardware device according to embodiments. For example, a computer may be understood as, but is not limited to, a concept including all of a smartphone, a tablet personal computer (PC), a desktop computer, a notebook, and a user client and an application running on each device.

Hereinafter, embodiments of the present invention will be described in detail with reference to the accompanying drawings.

Each step described in this specification is described as being performed by a computer, but the subject of each step is not limited thereto. At least a part of each step may be performed in different devices according to embodiments.

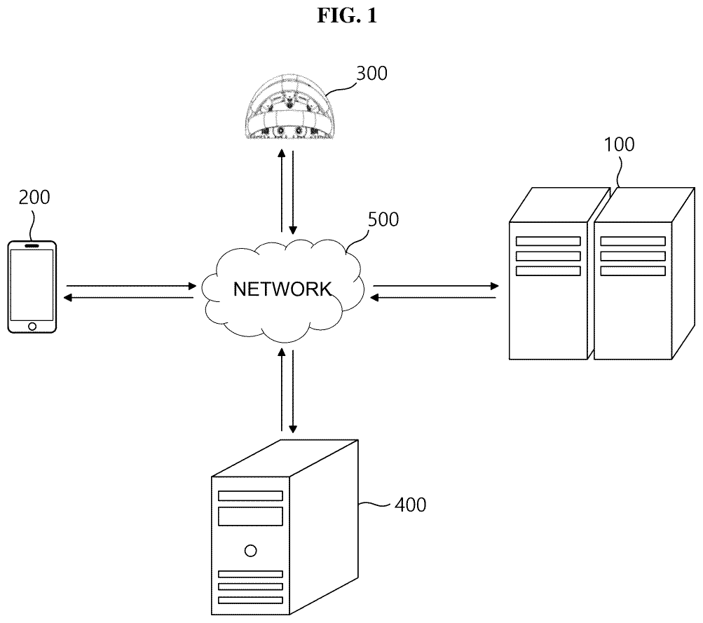

FIG. 1 is a diagram showing a system for providing therapy through brain wave data analysis according to an embodiment of the present invention.

Referring to FIG. 1, the system for providing therapy through brain wave data analysis according to the embodiment of the present invention may include a computing device 100, a user terminal 200, a therapy provision device 300, an external server 400, and a network 500.

Here, the system for providing therapy through brain wave data analysis shown in FIG. 1 is in accordance with the embodiment, and components thereof are not limited to the embodiment shown in FIG. 1 and may be added, changed, or removed as necessary.

According to the embodiment, the computing device 100 may perform a method of providing therapy through brain wave data analysis to provide therapy to a user. For example, the computing device 100 may be connected to the therapy provision device 300 via the network 500 to collect brain wave data of the user who is wearing the therapy provision device 300, may analyze the collected brain wave data to determine a therapy protocol, and may transmit a control command to control an operation in accordance with the determined therapy protocol to the therapy provision device 300 such that the therapy provision device 300 may provide therapy in accordance with the therapy protocol.

Although the computing device 100 has been described as being separately provided outside the therapy provision device 300 and controlling an operation of the therapy provision device 300 from the outside of the therapy provision device 300, this is merely illustrative, and the computing device 100 is not limited thereto. The computing device 100 may be embedded in the therapy provision device 300 to control an operation of the therapy provision device 300 in the therapy provision device 300.

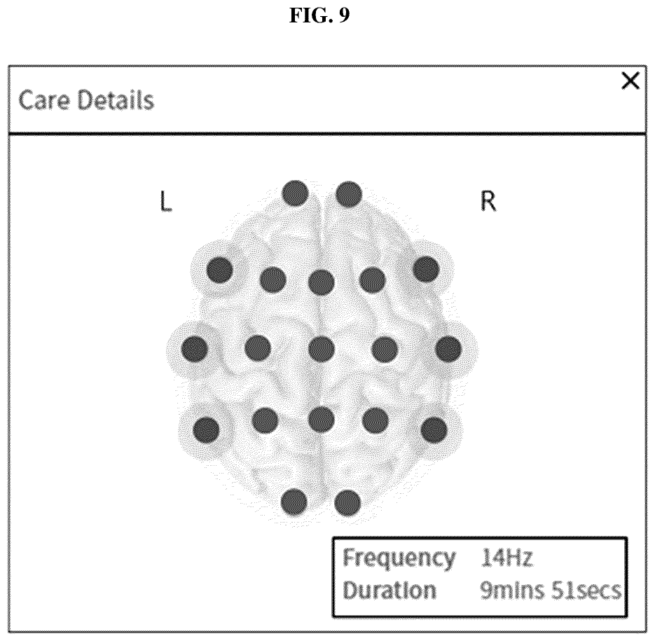

In various embodiments, the computing device 100 may be connected to the user terminal 200 via the network 500 and provide various information related to the method of providing therapy through brain wave data analysis to the user terminal 200. For example, the computing device 100 may provide a user interface (UI) (e.g., FIG. 9) that outputs the brain wave data collected from the user, information on analysis results of the brain wave data, and information on therapy derived through brain wave data analysis to the user terminal 200.

Here, the user terminal 200 may be any form of an entity in a system having a mechanism for communication with the computing device 100. For example, the user terminal 200 may be a PC, a notebook, a mobile terminal, a smartphone, a tablet PC, a wearable device, or the like and may be any type of terminal that may access a wireless/wired network. Also, the user terminal 200 may be any computing device that is implemented by at least one of an agent, an application programming interface (API), and a plug-in. In addition, the user terminal 200 may include an application source and/or a client application.

In various embodiments, the user terminal 200 may be a device for controlling an operation of the therapy provision device 300 and checking a data processing result of the computing device 100. For example, the user may receive a brain wave measurement protocol from the computing device 100 via the user terminal 200 to check the brain wave measurement protocol, may check whether brain wave measurement has been performed without errors in accordance with the brain wave measurement protocol, and may control whether to perform therapy which will be performed on the basis of a brain wave analysis result.

Here, the network 500 may be a connective structure in which nodes, such as a plurality of terminals and servers, may exchange information. For example, the network 500 may be a local area network (LAN), a wide area network (WAN), the Internet (World Wide Web (WWW)), a wired or wireless data communication network, a telephone network, a wired or wireless television communication network, or the like.

The wireless data communication network may be, but is not limited to, a 3rd generation (3G) network, a 4th generation (4G) network, a 5th generation (5G) network, a Third Generation Partnership Project (3GPP) network, a Fifth Generation Partnership Project (5GPP) network, a Long Term Evolution (LTE) network, a World Interoperability for Microwave Access (WiMAX) network, a Wi-Fi network, an Internet network, a LAN, a wireless LAN, a WAN, a personal area network (PAN), a radio frequency (RF) network, a Bluetooth network, a near-field communication (NFC) network, a satellite broadcasting network, an analog broadcasting network, a digital multimedia broadcasting (DMB) network, and the like.

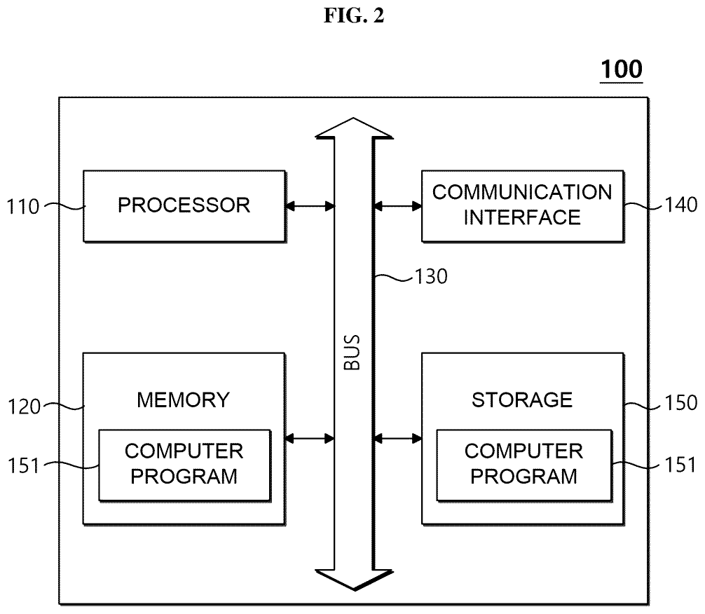

In the embodiment, the external server 400 may be connected to the computing device 100 via the network and may store and manage various information and data required for the computing device 100 to perform the method of providing therapy through brain wave data analysis or may receive, store, and manage various information and data that is derived when the computing device 100 performs the method of providing therapy through brain wave data analysis. For example, the external server 400 may be a storage server separately provided outside the computing device 100 but is not limited thereto. A hardware configuration of the computing device 100 that performs the method of providing therapy through brain wave data analysis will be described below with reference to FIG. 2.

FIG. 2 is a hardware configuration diagram of a computing device that performs a method of providing therapy through brain wave data analysis according to another embodiment of the present invention.

Referring to FIG. 2, in various embodiments, the computing device 100 may include at least one processor 110, a memory 120 into which a computer program 151 executed by the processor 110 is loaded, a bus 130, a communication interface 140, and a storage 150 in which the computer program 151 is stored. In FIG. 2, only components related to embodiments of the present invention are shown. Therefore, those skilled in the technical field to which the present invention pertains should appreciate that general-use components other than the components shown in FIG. 2 may be additionally included.

The processor 110 controls overall operations of each component of the computing device 100. The processor 110 may include a central processing unit (CPU), a micro-processor unit (MPU), a micro-controller unit (MCU), a graphics processing unit (GPU), or any form of processor well known in the technical field of the present invention.

In addition, the processor 110 may perform computation for at least one application or program for executing methods according to embodiments of the present invention, and the computing device 100 may include at least one processor.

In various embodiments, the processor 110 may further include a random access memory (RAM; not shown) and a read-only memory (ROM; not shown) which temporarily and/or permanently store a signal (or data) processed in the processor 110. Also, the processor 110 may be implemented in the form of a system on chip (SoC) including at least one of a GPU, a RAM, and a ROM.

The memory 120 stores various data, commands, and/or information. The computer program 151 may be loaded from the storage 150 into the memory 120 to perform methods/operations according to various embodiments of the present invention. When the computer program 151 is loaded into the memory 120, the processor 110 may perform the methods/operations by executing one or more instructions constituting the computer program 151. The memory 120 may be implemented as a volatile memory, such as a RAM, but the technical scope of the present disclosure is not limited thereto.

The bus 130 provides a communication function between the components of the computing device 100. The bus 130 may be implemented in one of various forms of buses such as an address bus, a data bus, a control bus, and the like.

The communication interface 140 supports wired or wireless Internet communication of the computing device 100. Also, the communication interface 140 may support various communication methods in addition to Internet communication. To this end, the communication interface 140 may include a communication module well known in the technical field of the present invention. In some embodiments, the communication interface 140 may be omitted.

The storage 150 may non-temporarily store the computer program 151. When the computing device 100 provides a process of providing therapy through brain wave data analysis, the storage 150 may store various information required for providing the process of providing therapy through brain wave data analysis.

The storage 150 may include a non-volatile memory, such as a ROM, an erasable programmable ROM (EPROM), an electrically erasable programmable ROM (EEPROM), a flash memory, and the like, a hard disk, a removable disk, or any form of computer-readable recording medium well known in the technical field to which the present invention pertains.

The computer program 151 may include one or more instructions that cause the processor 110 to perform methods/operations according to various embodiments of the present invention when loaded into the memory 120. In other words, the processor 110 may perform the methods/operations according to various embodiments of the present invention by executing the one or more instructions.

In the embodiment, the computer program 151 may include one or more instructions for performing a method of providing therapy through brain wave data analysis including a step of acquiring a user's brain wave data, a step of determining a therapy protocol on the basis of the acquired brain wave data, and a step of providing therapy to the user using the determined therapy protocol.

Steps of the methods or algorithms described in connection with embodiments of the present invention may be directly implemented using hardware, implemented using software modules executed by hardware, and implemented using a combination thereof. The software modules may reside in a RAM, a ROM, an EPROM, an EEPROM, a flash memory, a hard disk, a removable disk, a compact disc (CD)-ROM, or any form of computer-readable recording medium well known in the technical field to which the present invention pertains.

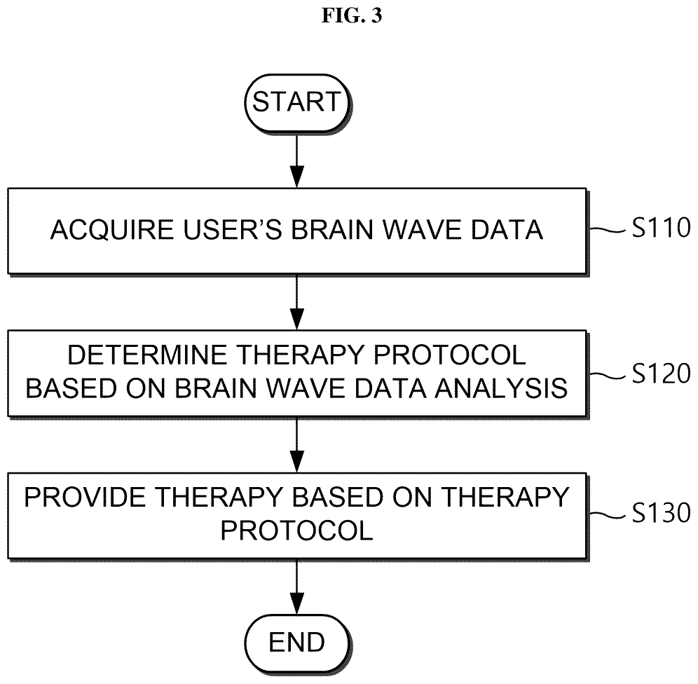

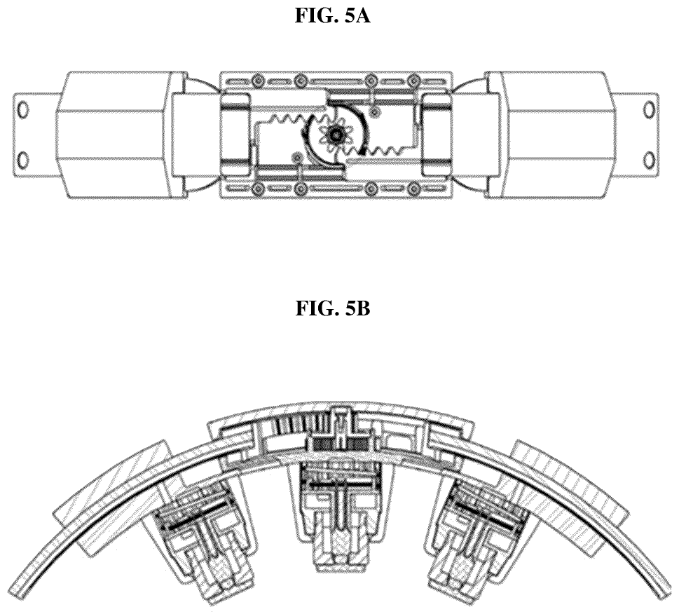

Components of the present invention may be implemented as a program (or application) and stored in a medium to be executed in combination with a computer which is hardware. Components of the present invention may be executed by software programming or software elements. Similarly, embodiments may be implemented in a programming or scripting language, such as C, C++, Java, assembler, or the like, to include various algorithms which are embodied as combinations of data structures, processes, routines, or other programming elements. Functional aspects may be implemented as an algorithm executed by one or more processors. A method of providing therapy through brain wave data analysis provided by the computing device 100 will be described below with reference to FIGS. 3 to 6.

FIG. 3 is a flowchart of a method of providing therapy through brain wave data analysis according to still another embodiment of the present invention.

Referring to FIG. 3, in step S110, the computing device 100 may acquire a user's brain wave data.

In various embodiments, the computing device 100 may be connected to the therapy provision device 300 via the network 500 and acquire the user's brain wave data that is measured using a brain wave measurement device included in the therapy provision device 300.

Here, the brain wave data may be a plurality of pieces of unit brain wave data (e.g., independent brain wave signals measured through channels) that are measured through a plurality of channels (e.g., 19 channels (e.g., Fp1, Fp2, F3, F4, C3, C4, P3, P4, O1, O2, F7, F8, T3, T4, T5, T6, Fz, Cz, and Pz) in total) that are included in the brain wave measurement device and attached to different positions on the user's head (scalp). For example, the therapy provision device 300 may include a brain wave measurement device including a plurality of channels arranged at positions consistent with a 10-20 system (10-20 electrode placement) and measure brain wave data including a plurality of pieces of unit brain wave data by measuring brain waves through the plurality of channels included in the brain wave measurement device, and the computing device 100 may acquire the brain wave data measured in accordance with the method.

In various embodiments, distances between the plurality of channels (the plurality of brain wave measurement channels) included in the brain wave measurement device may be adjusted in accordance with the user's head size. In other words, the distances between the plurality of channels may be instrumentally adjusted in accordance with the user's head size to maintain distance ratios between electrodes of the 10-20 system.

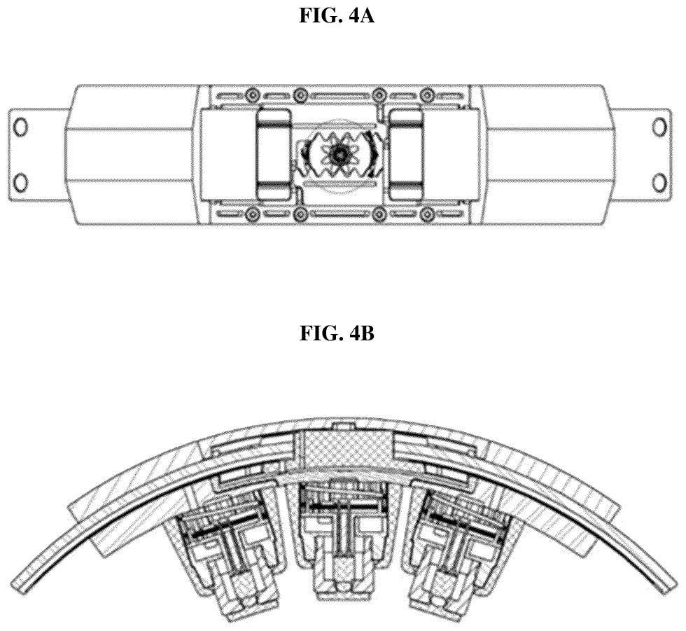

More specifically, as shown in FIGS. 4, 5 and 6, brain waves measured through the brain wave measurement device may be measured through channels kept with certain distance ratios. For example, the brain wave measurement device may have a structure in which distances between the channels for measuring brain waves may be kept constant, and thus can measure brain waves at accurate positions consistent with the 10-20 system while maintaining certain distance ratios between electrodes in accordance with the user's head size. In addition, the brain wave measurement device may match a position where a brain wave is measured to a position where therapy is provided, and provide the therapy to the accurate region that requires treatment.

In various embodiments, the computing device 100 may collect not only the user's brain wave data that is measured in a steady-state condition in which the user does not perform any action, but also brain wave data that is measured during a process of performing various tests on the user (e.g., a verbal fluency test, a Boston naming test, a mini-mental state examination (MMSE), a word list memory test, a construction praxis test, a word list recall test, a word list recognition test, a constructional recall test, a trail-making test A/B, and the like).

In various embodiments, the computing device 100 may process a plurality of pieces of brain wave data collected from a plurality of users to generate a plurality of pieces of quantified brain wave data (quantitative electroencephalogram (QEEG)).

In various embodiments, the computing device 100 may present a measurement protocol to the user terminal 200 and acquire brain wave data measured in accordance with the measurement protocol. More specifically, the computing device 100 may present information on a method of measuring brain waves, that is, a measurement protocol that is a guideline about how to measure brain waves.

As an example, the computing device 100 may present a measurement protocol for measuring brain waves with the eyes open for 30 seconds and then measuring brain waves with the eyes closed for 30 seconds.

As another example, the computing device 100 may determine a measurement protocol in accordance with requirements for a previously generated brain wave analysis model and propose the measurement protocol to the user. Here, the previously generated brain wave analysis model may change depending on a target setting. More specifically, when the previously generated brain wave analysis model is trained using 2-minute brain wave data of an Alzheimer's disease (AD) patient with the eyes closed as training data and a target set for the user is AD, the computing device 100 may determine a measurement protocol for measuring brain waves with the eyes closed for 2 minutes and provide the measurement protocol to the user.

Therefore, the computing device 100 may determine a measurement protocol to incorporate the requirements for brain wave data analysis and provide the measurement protocol to the user such that the user's brain waves may be measured for the purpose of therapy.

Here, the previously generated brain wave analysis model may be a brain wave analysis model that predicts a state from third brain wave data using brain wave data and labeled brain wave data as training data. For example, the brain wave analysis model may predict that the third brain wave data corresponds to AD.

Here, the brain wave analysis model (e.g., a neural network) may be composed of one or more network functions, which may be a set of calculation units generally referred to as “nodes” connected to each other. These “nodes” may also be referred to as “neurons.” The one or more network functions include one or more nodes. The nodes (or neurons) constituting the one or more network functions may be connected to each other through one or more “links.”

In the brain wave analysis model, the one or more nodes connected through the links may have a relationship between an input node and an output node. The concepts of an input node and an output node are relative. Any node which is an output node relative to one node may be an input node relative to another node, and vice versa. As described above, the relationship between an input node and an output node may be established on the basis of a link. One input node may be connected to one or more output nodes through links, and vice versa.

In the relationship between an input node and an output node connected through one link, a value of the output node may be determined on the basis of data input to the input node. Here, a node connecting the input node and the output node to each other may have a weight. The weight may be variable and varied by the user or an algorithm to perform a function required by the brain wave analysis model. For example, when one or more input nodes and one output node are connected to each other through separate links, the output node may determine a value of the output node on the basis of values input to the input nodes connected to the output node and weights set for the links corresponding to each of the input nodes.

As described above, in the brain wave analysis model, one or more nodes are connected to each other through one or more links to have a relationship between an input node and an output node. Characteristics of the brain wave analysis model may be determined in accordance with the number of nodes, the number of links, relationships between the nodes and the links, and weight values assigned to each of the links in the brain wave analysis model. For example, when two brain wave analysis models have the same number of nodes, the same number of links, and different weights for identical links, the two brain wave analysis models may be recognized to be different.

Some nodes constituting the brain wave analysis model may constitute one layer on the basis of distances from an initial input node. For example, a set of nodes having a distance of n from the initial input node may constitute layer n. The distance from the initial input node may be defined in accordance with the minimum number of links which should be passed through to reach a corresponding node from the initial input node. However, this definition of a layer is for description, and the order of a layer in the brain wave analysis model may be defined in accordance with a different method from that described above. For example, a layer of nodes may be defined in accordance with a distance from a final output node.

The initial input node may be one or more nodes to which data is directly input without passing through any link in the relationships with other nodes among nodes in the brain wave analysis model. Alternatively, the initial input node may be nodes that do not have other input nodes connected through links in the link-based relationships between nodes within the brain wave analysis model. Similarly, the final output node may be one or more nodes which have no output node in the relationships with other nodes among the nodes in the brain wave analysis model. Also, hidden nodes may be nodes constituting the brain wave analysis model other than the initial input node and the final output node. In the brain wave analysis model according to an embodiment of the present invention, the number of nodes in the input layer may be larger than the number of nodes in a hidden layer close to the output layer, and the number of nodes may decrease from the input layer to the hidden layer.

The brain wave analysis model may include one or more hidden layers. Hidden nodes of the hidden layers may use outputs of the previous layer and outputs of surrounding hidden nodes as inputs. The number of hidden nodes in each hidden layer may be the same as or different from the number of hidden nodes in other hidden layers. The number of nodes in the input layer may be determined on the basis of the number of data fields of input data and may be the same as or different from the number of hidden nodes. The input data input to the input layer may be computed by the hidden nodes of the hidden layers, and a computation result may be output by a fully connected layer (FCL) which is the output layer.

In various embodiments, the brain wave analysis model may be a deep learning model.

The deep learning model (e.g., a deep neural network (DNN)) may be a brain wave analysis model including a plurality of hidden layers in addition to an input layer and an output layer. When the DNN is used, latent structures of data may be determined. In other words, latent structures of photos, text, videos, voice, and music (e.g., what objects are in the photos, what the content and feelings of the text are, what the content and feelings of the voice are, and the like) may be determined.

The DNN may be, but is not limited to, a convolutional neural network (CNN), a recurrent neural network (RNN), an autoencoder, a generative adversarial network (GAN), a restricted Boltzmann machine (RBM), a deep belief network (DBN), a Q network, a U network, a Siamese network, and the like.

In various embodiments, the network functions may include an autoencoder. Here, the autoencoder may be an artificial neural network for outputting output data similar to input data.

The autoencoder may include at least one hidden layer, and an odd number of hidden layers may be disposed between input and output layers. The number of nodes in each layer may decrease from the input layer to an intermediate layer called a bottleneck layer (encoding), and then increase from the bottleneck layer to the output layer (symmetrical to the input layer) symmetrically to the decrease. The number of nodes of a dimension reduction layer and the number of nodes of a dimension restoration layer may or may not be symmetrical. In addition, the autoencoder may perform non-linear dimension reduction. The number of input and output layers may correspond to the number of sensors remaining after preprocessing of input data. The autoencoder may have a structure in which the number of nodes in a hidden layer included in the encoder decreases with an increase in the distance from the input layer. When the number of nodes in the bottleneck layer (a layer having a smallest number of nodes positioned between an encoder and a decoder) is too small, a sufficient amount of information may not be delivered, and thus the number of nodes in the bottleneck layer may be maintained to be a specific number or more (e.g., half the number of nodes in the input layer or more).

In step S120, the computing device 100 may analyze the brain wave data acquired in step S110 to determine a therapy protocol for the user.

In various embodiments, when it is determined by analyzing the user's brain wave data that it is necessary to provide therapy to the user, the computing device 100 may determine a therapy protocol for the user.

Here, the therapy protocol is for determining therapy that will be provided to the user. For example, the therapy protocol may include sessions (e.g., a period and number) of the therapy that will be provided to the user, a frequency of the therapy, a treatment area of the therapy, a duration of the therapy, and a light wavelength of the therapy. For example, the therapy protocol may be determined in the form of “(12 times in 4 weeks), (13 Hz to 14 Hz), (frontal, global, and temporal regions), (10 minutes), (near infrared wavelength)” but is not limited thereto.

More specifically, first, the computing device 100 may determine the necessity to provide therapy to the user by analyzing the user's brain wave data.

As an example, the computing device 100 may determine whether the user has a brain disease by analyzing the user's brain wave data, and when it is determined that the user has a brain disease, may determine that it is necessary to provide therapy to the user.

As another example, the computing device 100 may compare the user's brain wave data with brain wave data of normal people having the same attributes (e.g., age, sex, and the like), and when there is an error exceeding a predetermined range between the user's brain wave data and the normal people's brain wave data, may determine that it is necessary to provide therapy to the user.

As still another example, the computing device 100 may analyze the user's brain wave data, and when an abnormal region is specified, may determine that it is necessary to provide therapy to the user. Here, the abnormal region may be, but is not limited to, an abnormal brain area or a brain area associated with abnormal brain waves on the basis of the brain wave data.

In various embodiments, the computing device 100 may specify an abnormal region by analyzing the user's brain wave data on the basis of a user profile.

The user profile may include information on the user such as the user's age, sex, dominant hand, cognitive scores (such as an MMSE score and the like), existing brain wave measurement results, statistical values of the existing brain wave measurement results, and the like. For example, the computing device 100 may calculate a statistical value (e.g., an average) of brain wave data of normal people having the same profile as the user to set position-specific brain wave reference values of normal people, compare the position-specific brain wave reference values of normal people with position-specific brain wave values included in the brain wave data, and specify, as an abnormal region, an area corresponding to a position where a difference between a brain wave value and a brain wave reference value is a preset threshold or more.

In various embodiments, the computing device 100 may specify an abnormal region of the user on the basis of a preset target. For example, the computing device 100 may set a target in advance, compare the position-specific brain wave values included in the brain wave data with position-specific brain wave reference values of the target, and specify, as an abnormal region, an area corresponding to a position where a difference between a brain wave value and a brain wave reference value is a preset threshold or more.

Here, the target is a purpose for which therapy will be provided, a disease to be treated through the therapy, and/or an effect to be achieved through the therapy. For example, the target may be, but is not limited to, a specific brain disease (e.g., AD, Lewy body dementia, Parkinson's disease, a stroke, attention deficit hyperactivity disorder (ADHD), depression, memory deficits, traumatic brain injury, or the like) and/or health improvement such as treatment of a specific brain disease, healthcare, normal brain waves, and the like. For example, when the preset target is “normal brain waves,” the computing device 100 may set position-specific brain wave reference values using position-specific brain wave values included in normal brain wave data and compare the position-specific brain wave reference values which are set using the normal brain wave data with the position-specific brain wave values based on the user's brain wave data to specify an abnormal position.

In various embodiments, the computing device 100 may specify an abnormal region on the basis of the user's measured brain wave history. For example, when first to n^{th} brain waves correspond to the user's resting state, the computing device 100 may specify an abnormal region on the basis of the user's brain wave history using the user's first to n^{th} brain waves. In addition, the computing device 100 may specify an abnormal region on the basis of the user's measured brain wave history only when the user's measured brain wave history is from a specific time period. For example, when the user's brain waves have been measured 5 times at the age of 59 and 5 times at the age of 60 and then the user's brain wave history is used at the age of 60 to specify an abnormal region, the computing device 100 may specify an abnormal region on the basis of only the brain wave data measured 5 times at the age of 60. However, the computing device 100 may specify an abnormal region on the basis of brain wave data measured in a certain time period rather than brain wave data measured at a certain age.

Subsequently, when it is necessary to provide therapy to the user, the computing device 100 may determine a therapy protocol.

In various embodiments, when an abnormal region is specified on the basis of the brain wave data, the computing device 100 may determine, as a therapy protocol for the user, a frequency and a duration of therapy for leading a brain wave corresponding to the specified abnormal region to a preset reference value. For example, when a target set for the user is AD and areas specified as abnormal regions are areas 01 and 02, the computing device 100 may determine a frequency and a duration (e.g., 10 Hz, 3 minutes) of therapy for the purpose of leading brain waves from areas 01 and 02 of the AD patient to normal-level values.

In various embodiments, the computing device 100 may determine a therapy protocol for the user on the basis of the user's brain disease severity. For example, the computing device 100 may specify the user's abnormal region by analyzing the user's brain wave data. When an abnormal region of the user's brain is specified, the computing device 100 may analyze a brain wave corresponding to the abnormal region to calculate an indicator (e.g., a z-score) corresponding to a degree of abnormality of the abnormal region and may determine a therapy protocol for the user on the basis of the indicator corresponding to the degree of abnormality of the abnormal region, that is, determine a therapy protocol for the user on the basis of the degree of abnormality of the abnormal region. For example, when the degree of alpha slowing is high (e.g., a z-score of −1 or less) in the abnormal region, the frequency of the therapy may be determined to be 15 Hz as the therapy protocol but is not limited thereto.

In various embodiments, the user's brain wave data may be analyzed to determine a treatment area of the therapy as the therapy protocol for the user.

In various embodiments, the computing device 100 may determine a treatment area as the therapy protocol for the user on the basis of the preset target. As an example, the computing device 100 may define target-specific treatment areas in advance, and when the target is set by the user to whom the therapy will be provided, may determine a treatment area as the therapy protocol on the basis of the predefined target-specific treatment areas. For example, when the preset target is AD and the abnormal region is associated with a default mode network (DMN), the computing device 100 may determine the medial prefrontal cortex, the precuneus, the posterior cingulate cortex, the inferior parietal lobe, and the hippocampus as treatment areas.

In various embodiments, the computing device 100 may analyze the user's brain wave data to diagnose the user's brain disease and determine a treatment area on the basis of a brain disease diagnosis result. For example, when it is determined that the user has a specific brain disease by analyzing the user's brain wave data, the computing device 100 may determine a region corresponding to the specific brain disease as a first treatment region and determine, as a second treatment region, a region related to treatment of the specific brain disease, that is, a region where there is an effect when the therapy is provided for the specific brain disease (or a disease group including the brain disease).

In various embodiments, when an abnormal region of the user is specified by analyzing the user's brain wave data, the computing device 100 may determine a therapy protocol such that the therapy may be provided only to the specified abnormal region.

In various embodiments, when two or more abnormal regions are specified at two or more different positions of the single user's brain by analyzing the user's brain wave data, the computing device 100 may separately determine a therapy protocol for each of the two or more abnormal regions such that individual therapy may be provided to the two or more abnormal regions. However, the present invention is not limited thereto, and when two or more abnormal regions are specified at two or more different positions of the user's brain, the computing device 100 may determine a common therapy protocol corresponding to the two or more abnormal regions.

In step S130, the computing device 100 may provide the therapy to the user using the therapy protocol determined through step S120.

Here, the therapy may be photobiomodulation (PBM). PBM may be provided using various wavelengths such as a wavelength of 620 nm to 780 nm, a wavelength of 780 nm to 1400 nm, or the like. PBM can increase adenosine triphosphate (ATP) synthesis and oxygen consumption at the cellular level and improve mitochondrial metabolism in a living body. In addition, PBM preferably aims to promote the growth and healing of neuronal cells and ameliorate brain disorders through a gene transcription process.

In various embodiments, the computing device 100 may be connected to the therapy provision device 300 via the network 500 and provide a control command determined on the basis of the therapy protocol to the therapy provision device 300 such that the therapy provision device 300 may perform an operation of providing the therapy in accordance with the control command. For example, when the therapy protocol determined by the computing device 100 is “(4 weeks 12 times), (13 Hz to 14 Hz), (frontal, global, and temporal regions), (10 minutes), (near infrared wavelength),” the therapy provision device 300 may provide therapy of outputting a near infrared wavelength with a frequency of 13 Hz to 14 Hz to the frontal, global, and temporal regions for 10 minutes 12 times in 4 weeks on the basis of the control command received from the computing device 100.

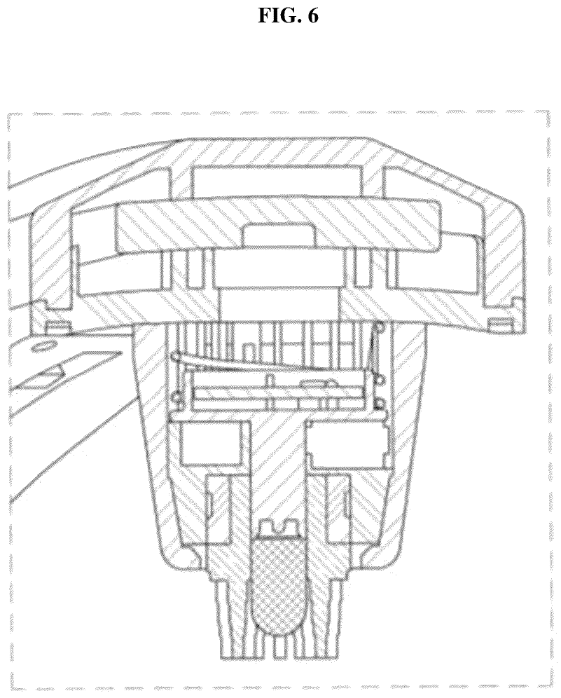

In various embodiments, the computing device 100 may provide the therapy at a position corresponding to a channel for measuring a brain wave through the therapy provision device 300. For example, as shown in FIG. 6, the computing device 100 may place an electrode of the therapy provision device 300 and a PBM provision module at the same position to provide the therapy at a position corresponding to a channel for measuring a brain wave. In this way, the computing device 100 provides the therapy at a region (treatment area) of the user requiring treatment through the therapy provision device 300, thereby performing measurement and providing treatment simultaneously.

This therapy provision method may change in accordance with severity of a patient, a corresponding disease, whether there is an accompanying disease, history of therapy provision, and the like. In addition, regardless of the determination of a treatment area, the computing device 100 may determine to provide therapy to the user in accordance with a treatment area and a treatment method that are manually set on the basis of a controller's design.

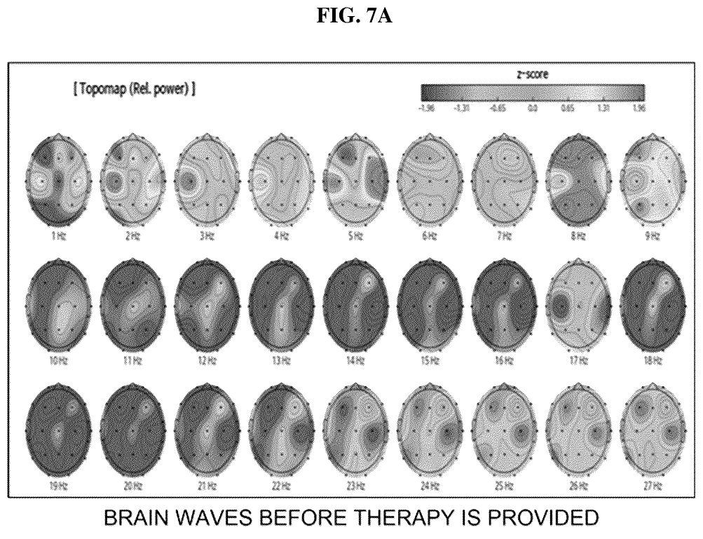

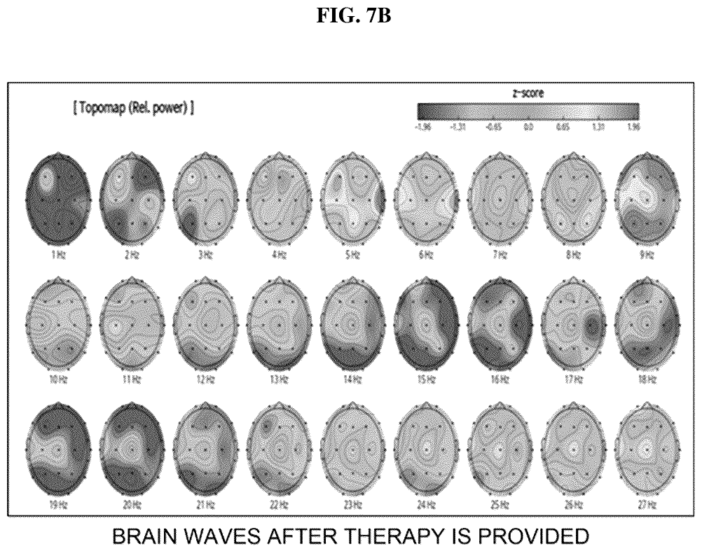

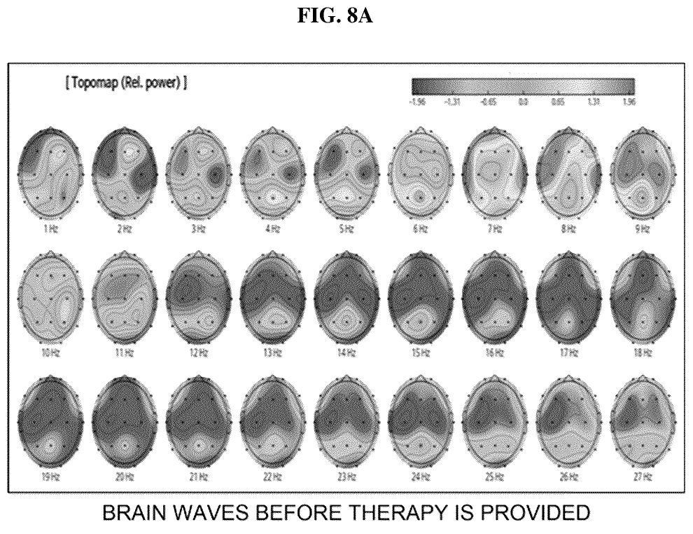

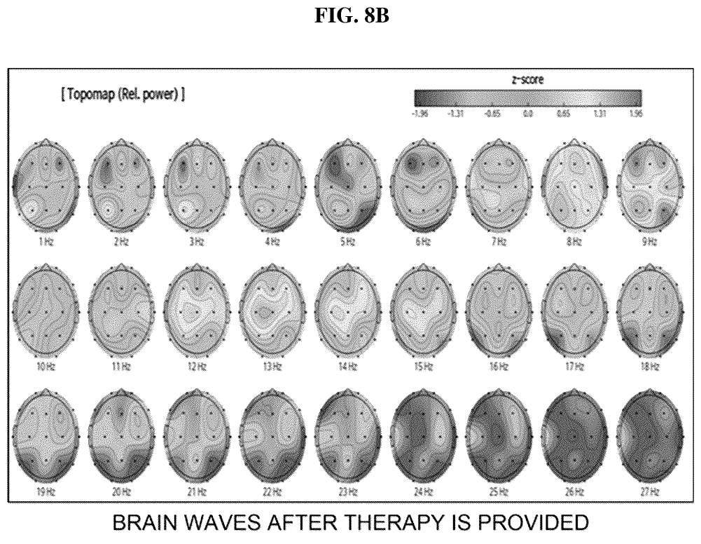

FIGS. 7 and 8 are sets of brain wave views for comparing brain waves before and after therapy is provided using a method of providing therapy through brain wave data analysis according to various embodiments of the present invention.

First, a first patient who had weak brain waves in the alpha region and significantly weak power in the lateral sides in general as shown in FIG. 7A was treated with therapy of outputting PBM at 13 Hz over the entire brain 12 times in total for 4 weeks, resulting in increased power in the sensorimotor rhythm (SMR) region, enhanced alpha power in the temporal region, and increased frequency of alpha brain waves as shown in FIG. 7B.

Also, a second patient who had weaker power in the temporal region in general than the first patient as shown in FIG. 8A was treated with therapy of outputting PBM at 14 Hz to the temporal region alone for 4 weeks and then outputting PBM at 14 Hz and PBM at 40 Hz of the gamma region over the entire brain for the next 4 weeks, resulting in increased power in the SMR region, enhanced alpha power in the temporal region, and increased frequency of alpha brain waves as shown in FIG. 8B.

According to an embodiment, as shown in FIGS. 7 and 8, when therapy is provided to a user of the present invention, the SMR region or alpha power is improved, which improves a user's sleeping problems. In other words, unlike conventional SMR neurofeedback causing a user to solve a specific problem and improve sleeping problems, the present invention provides direct therapy to improve sleeping problems.

The method of providing therapy through brain wave analysis has been described above with reference to the flowchart shown in the drawing. While the method of providing therapy through brain wave analysis has been shown and described as a series of blocks for the purpose of simplicity of explanation, the present invention is not limited to the order of blocks. Some blocks may be performed concurrently or in an order different from that shown and described herein. Also, a new block which is not described herein or shown in the drawing may be added, or some blocks may be removed or changed.

Embodiments of the present invention have been described above with reference to the accompanying drawings, but those skilled in the technical field to which the present invention pertains should understand that the present invention can be implemented in other specific forms without changing the technical spirit or essential features thereof. Therefore, it should be understood that the embodiments described above are illustrative in all aspects and are not restrictive.

### DESCRIPTION OF SIGNS

100: computing device
200: user terminal
300: therapy provision device
400: external server
500: network

1. A method of providing therapy through brain wave analysis performed by a computing device, the method comprising:
acquiring a user's brain wave data;
determining a therapy protocol on the basis of the acquired brain wave data, and
providing therapy to the user using the determined therapy protocol.

2. The method of claim 1, wherein the determining of the therapy protocol comprises:
analyzing the acquired brain wave data to determine necessity of providing therapy to the user; and
determining a therapy protocol for the user when it is determined that it is necessary to provide therapy to the user, the determined therapy protocol including at least one of sessions, a frequency, a treatment area, a duration time, and a light wavelength of the therapy to be provided to the user.

3. The method of claim 1, wherein the determining of the therapy protocol comprises:
specifying an abnormal region on the basis of the acquired brain wave data; and
determining a frequency and a duration of the therapy for leading a brain wave corresponding to the specified abnormal region to a preset reference value as a therapy protocol for the user.

4. The method of claim 3, wherein the specifying of the abnormal region comprises:
setting a target; and
comparing position-specific brain wave values included in the acquired brain wave data with position-specific brain wave reference values in accordance with the set target to specify the abnormal region.

5. The method of claim 1, wherein the determining of the therapy protocol comprises determining a treatment area of the therapy as the therapy protocol for the user by analyzing the acquired brain wave data.

6. The method of claim 5, wherein the determining of the treatment area comprises, when the user is diagnosed with a specific brain disease by analyzing the acquired brain wave data, determining a region in which the specific brain disease is diagnosed as a first treatment area and determining a region related to treatment of the diagnosed specific brain disease as a second treatment area.

7. The method of claim 1, wherein the determining of the therapy protocol comprises, when an abnormal region of the user is specified by analyzing the acquired brain wave data, determining the therapy protocol such that the therapy is provided to the specified abnormal region alone.

8. The method of claim 1, wherein the determining of the therapy protocol comprises, when two or more abnormal regions of the user are specified by analyzing the acquired brain wave data, separately determining therapy protocols corresponding to each of the two or more specified abnormal regions.

9. The method of claim 1, wherein the determining of the therapy protocol comprises:
specifying an abnormal region of the user by analyzing the acquired brain wave data;
calculating an indicator corresponding to a degree of abnormality of the specified abnormal region on the basis of brain waves corresponding to the specified abnormal region; and
determining a frequency of the therapy as the therapy protocol for the user on the basis of the calculated indicator.

10. The method of claim 1, wherein the acquiring of the brain wave data comprises acquiring the user's brain wave data measured through a plurality of channels included in a brain wave measurement device in accordance with a preset measurement protocol, and
distances between the plurality of channels may be adjusted in accordance with the user's head size.

11. A computing device for performing a method of providing therapy through brain wave data analysis, the computing device comprises:
a processor;
a network interface;
a memory; and
a computer program which is loaded into the memory and executed by the processor,
wherein the processor performs the method of claim 1 by executing one or more instructions included in the computer program.

12. A non-transitory recording medium readable by a computing device that is combined with a computing device and on which a computer program for performing the method of claim 1.
